(19) United States
(12) Patent Application Publication
(10) Pub. No.: US 2026/0263131 A1
(43) Pub. Date: Sep. 10, 2026

(54) INTRAMEDULLARY NAIL HAVING INTEGRATED COMPRESSION MECHANISM, AND RELATED INSTRUMENTATION, SYSTEMS, AND METHODS

(71) Applicant: DePuy Synthes Products, Inc., Raynham, MA (US)

(72) Inventor: Kevin Clancy, West Chester, PA (US)

(21) Appl. No.: 19/550,710

(22) Filed: Feb. 26, 2026

Related U.S. Application Data
(60) Provisional application No. 63/766,438, filed on Mar. 4, 2025.

(51) Int. Cl. A61B 17/88; A61B 17/72; A61B 90/00; A61B 17/00; A61B 17/68

(52) U.S. Cl. CPC A61B 17/8872; A61B 17/7225; A61B 17/7291; A61B 90/03; A61B 2017/00367; A61B 2017/00867; A61B 2017/681; A61B 2090/034

(57) ABSTRACT

An intramedullary nailing system has an intramedullary nail, an inserter, and a deployment instrument. The nail has an elongate body housing a spring and a shuttle that is joined with the spring and defining an aperture for receiving a locking member for affixing the shuttle to bone. The inserter has a leading end for coupling with the nail, a trailing end, and a cannulation extending forwardly from the trailing to leading end. The instrument has an elongate body having trailing, intermediate, and leading portions. The leading portion is configured to extend through the cannulation and couple with the shuttle. An actuator defines transmission formations for engaging complementary structures of the intermediate portion. The actuator is manipulatable to cause the transmission formations to engage the complementary structures for translating the instrument body and shuttle rearwardly together, thereby loading a forward spring force in the spring.

### CROSS-REFERENCE TO RELATED APPLICATIONS

The present application claims the benefit of priority to U.S. Provisional Application No. 63/766,438, filed Mar. 4, 2025, the entire contents of which are incorporated herein by reference.

### TECHNICAL FIELD

The present invention relates to bone implants, and more particularly to intramedullary nails and instrumentation and related surgical systems for implanting intramedullary nails.

### BACKGROUND

Conventional intramedullary nails are configured to be inserted into the medullary canal of a bone that has been fractured so as to define a proximal bone segment and a distal bone segment that is separated from the proximal bone segment by a bone gap. Conventional intramedullary nails are elongate along a substantially central longitudinal axis, and include bone anchor holes that are configured to receive bone anchors. The bone anchor holes can include proximal bone anchor holes that extend through the proximal end of the intramedullary nail and distal bone anchor holes that extend through the distal end of the intramedullary nail. Thus, the intramedullary nail can be inserted into the medullary canal of the fractured long bone such that the proximal bone anchor holes are aligned with the proximal bone segment and the distal bone anchor holes are aligned with the distal bone segment on opposite sides of the bone gap.

The bone segments can be positioned by a surgeon and the bone screws can be driven into the bone segments and the corresponding bone anchor holes so as to fasten the intramedullary nail to the fractured long bone and stabilize the proximal and distal bone segments relative to each other, thereby promoting healing. The relative positions of the bone segments are fixed once the bone screws are set using such traditional designs. Therefore, an improved intramedullary nail that compresses the bone segments against each other during the healing process is desired.

### SUMMARY

According to an embodiment of the present disclosure, an instrument assembly includes an intramedullary (IM) nail, a nail inserter, and an instrument. The IM nail has a nail body elongate along a longitudinal nail axis, a spring member disposed within the nail body, and a shuttle joined with the spring member. The shuttle defines an aperture that extends through the shuttle along an aperture axis angularly offset from the longitudinal nail axis. The aperture is configured to receive a locking member for affixing the shuttle to bone. The nail inserter has an inserter body that defines a trailing end, an opposed leading end, and a cannulation extending from the trailing end to the leading end in a forward direction oriented along a central axis of the inserter body. The leading end is configured to couple with a trailing end of the IM nail. The instrument includes an elongate instrument body, a first force actuator, and a second force actuator. The instrument body has a leading portion that defines engagement structures and is configured to extend through the cannulation of the nail inserter and into an opening at a trailing end of the shuttle to position the engagement structures adjacent complementary engagement formations defined along a trailing portion of the shuttle. The first force actuator is configured to move the instrument body relative to the nail inserter to cause the engagement structures to engage the complementary engagement formations of the shuttle, thereby attaching the instrument to the shuttle. The instrument body also has an intermediate portion that defines one or more transmission structures. The second force actuator is connectable to the intermediate portion of the instrument body. The second force actuator defines one or more complementary transmission formations configured to engage the one or more transmission structures. The second force actuator is manipulatable to cause the one or more transmission formations to engage the one or more transmission structures in a manner to translate the instrument body and the shuttle in unison in a rearward direction opposite the forward direction, thereby displacing the spring member so as to load a spring force in the spring member in the forward direction.

According to another embodiment of the present disclosure, an intramedullary nailing system includes an intramedullary (IM) nail, an inserter, and a deployment instrument. The IM nail has an elongate body that houses a spring and a shuttle that is joined with the spring. The shuttle defines an aperture for receiving a locking member for affixing the shuttle to bone. The inserter has a leading end (for coupling with the IM nail), a trailing end, and a cannulation extending forwardly from the trailing to leading end. The instrument has an elongate body having trailing, intermediate, and leading portions. The leading portion is configured to extend through the cannulation and couple with the shuttle. An actuator defines transmission formations for engaging complementary structures of the intermediate portion. The actuator is manipulatable to cause the transmission formations of the actuator to engage the complementary structures of the intermediate portion of the instrument body for translating the instrument body and shuttle rearwardly in unison, thereby loading a forward spring force in the spring.

According to an additional embodiment of the present disclosure, a method for preparing an intramedullary (IM) nail to provide bone compression includes a step of attaching a leading portion of a nail inserter to a trailing portion of a nail body of the IM nail. The nail body is elongate along a longitudinal nail axis and defines a leading portion spaced from the trailing portion in a forward direction. The IM nail includes a spring member joined with a shuttle within an interior channel defined by the nail body. The shuttle defines at least one aperture configured to receive a locking member for affixing the shuttle to bone. The method includes a step of advancing an instrument body of an instrument through a longitudinal cannulation of the nail inserter and into engagement with the shuttle and a step of coupling a leading portion of the instrument body with the shuttle. The method also includes a step of manipulating a transmission member of the instrument relative to the instrument body in a manner translating the instrument body and the shuttle in unison in a rearward direction opposite the forward direction, thereby loading a spring force in the spring member in the forward direction, wherein the spring force is configured to drive the shuttle in the forward direction to provide the bone compression.

### BRIEF DESCRIPTION OF THE DRAWINGS

The foregoing summary, as well as the following detailed description of illustrative embodiments of the present application, will be better understood when read in conjunction with the appended drawings. For the purposes of illustrating the features of the present application, there is shown in the drawings illustrative embodiments. It should be understood, however, that the application is not limited to the precise arrangements and instrumentalities shown. In the drawings:

FIG. 1 is a side plan view of an intramedullary (IM) nailing system having an IM nail, a nail inserter, and a deployment instrument for deploying a compression mechanism of the IM nail, according to an embodiment of the present disclosure;

FIG. 2A is a perspective view of the IM nail illustrated in FIG. 1;

FIGS. 2B-2C are sectional side views of the IM nail illustrated in FIG. 2A;

FIG. 3A is a perspective view of the compression mechanism of the IM nail shown in FIGS. 2A-2C, according to an embodiment of the present disclosure;

FIG. 3B is an exploded perspective view of the compression mechanism illustrated in FIG. 3A;

FIGS. 3C-3D are perspective side views of the compression mechanism shown in FIGS. 3A-3B, shown in a first configuration (FIG. 3C) and in a second, loaded configuration (FIG. 3D);

FIGS. 4A-4B are side views of a trailing portion of the IM nail illustrated in FIGS. 2A-2C, shown in the first configuration (FIG. 4A) and in the second, loaded configuration (FIG. 4B);

FIG. 5A is a perspective view of the nail inserter illustrated in FIG. 1, according to an embodiment of the present disclosure;

FIG. 5B is a sectional side view of the nail inserter illustrated in FIG. 5A;

FIG. 5C is a sectional side view of a leading portion of the nail inserter coupled with a trailing portion of the IM nail, according to an embodiment of the present disclosure;

FIG. 6A is a perspective view of the deployment instrument illustrated in FIG. 1, according to an embodiment of the present disclosure;

FIG. 6B is a sectional side view showing the deployment instrument of FIG. 6A extending through the nail inserter of FIGS. 5A-5C and coupled with the IM nail of FIGS. 2A-2C while in the first configuration, according to an embodiment of the present disclosure;

FIG. 6C is an enlarged view of a portion of the deployment instrument and an adjacent portion of the nail inserter illustrated in the first configuration in FIG. 6B;

FIG. 6D is a sectional side view showing the deployment instrument of FIG. 6A extending through the nail inserter of FIGS. 5A-5C and coupled with the IM nail of FIGS. 2A-2C while in the second configuration, according to an embodiment of the present disclosure;;

FIG. 6E is an enlarged view of a portion of the deployment instrument and an adjacent portion of the nail inserter illustrated in the second configuration in FIG. 6D;

FIG. 6F is a perspective view of the deployment instrument illustrated in FIG. 6A, showing an additional force multiplier component for use with the instrument, according to an embodiment of the present disclosure;

FIG. 7 is a diagram view showing a method for using the IM nailing system to prepare an IM nail for implantation, according to an embodiment of the present disclosure; and

FIG. 8 is a diagram view showing a method for using the IM nailing system to implant the IM nail, according to an embodiment of the present disclosure.

### DETAILED DESCRIPTION OF ILLUSTRATIVE EMBODIMENTS

The present disclosure can be understood more readily by reference to the following detailed description taken in connection with the accompanying figures and examples, which form a part of this disclosure. It is to be understood that this disclosure is not limited to the specific devices, methods, applications, conditions or parameters described and/or shown herein, and that the terminology used herein is for the purpose of describing particular embodiments by way of example only and is not intended to be limiting of the scope of the present disclosure. Also, as used in the specification including the appended claims, the singular forms “a,” “an,” and “the” include the plural, and reference to a particular numerical value includes at least that particular value, unless the context clearly dictates otherwise.

The term “plurality”, as used herein, means more than one. When a range of values is expressed, another embodiment includes from the one particular value and/or to the other particular value. Similarly, when values are expressed as approximations, by use of the antecedent “about,” it will be understood that the particular value forms another embodiment. All ranges are inclusive and combinable.

The terms “approximately”, “about”, and “substantially”, as used herein with respect to dimensions, angles, ratios, and other geometries, take into account manufacturing tolerances. Further, the terms “approximately”, “about”, and “substantially” can include 10% greater than or less than the stated dimension, ratio, or angle. Further, the terms “approximately”, “about”, and “substantially” can equally apply to the specific value stated.

It should be understood that, although terms involving numerical prepositions (e.g., “first,” “second,” “third,” etc.) can be used herein to describe various features, such features should not be limited by these terms. These terms are instead used to distinguish one feature from another. For example, a “first” element could be termed a “second” element in another context, and, similarly, a “second” element could be termed a “first” element in another context, without departing from the scope of the embodiments disclosed herein.

The embodiments disclosed herein pertain to surgical systems and instrumentation for implanting an intramedullary (IM) nail into patient anatomy. For example, the features disclosed herein can be configured for implanting an IM nail in an anatomical proximal direction through the calcaneus, talus, and into the tibial medullary canal during a Tibio-Talo-Calcaneal (TTC) ankle fusion procedure, particularly in a manner that allows the IM nail to provide intra- and post-operation compression between the tibia and calcaneus. Although the following disclosure describes the features herein in the context of a TTC ankle fusion procedure, it should be appreciated that the embodiments, IM nails, instrumentation, and surgical system(s) described herein can be adapted for use in various other surgical procedures for treating bone-related injuries and conditions.

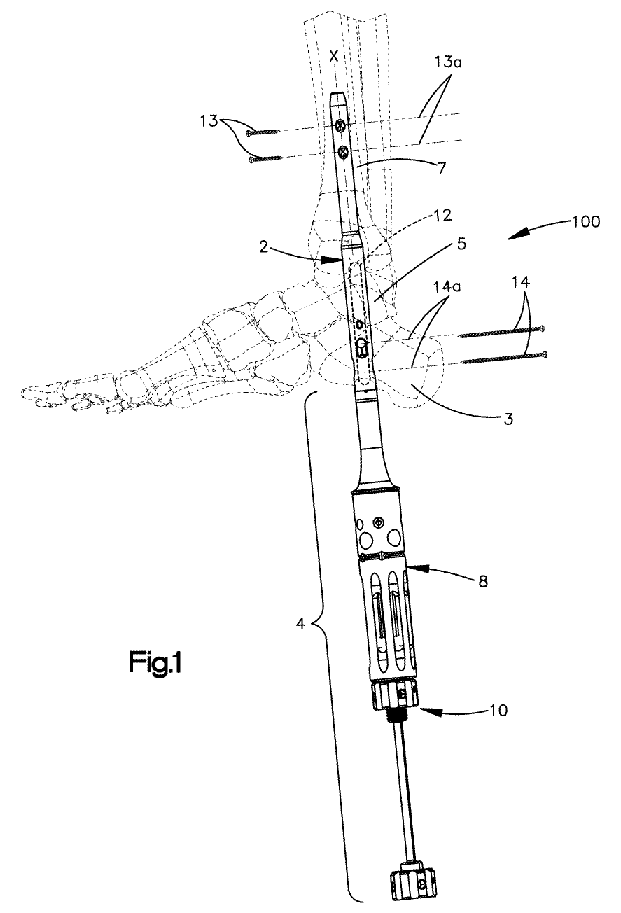

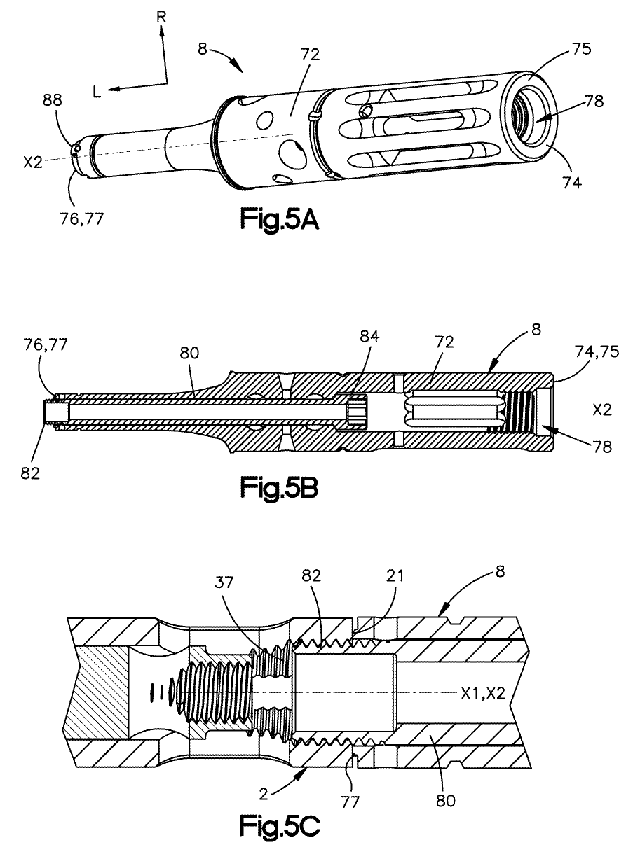

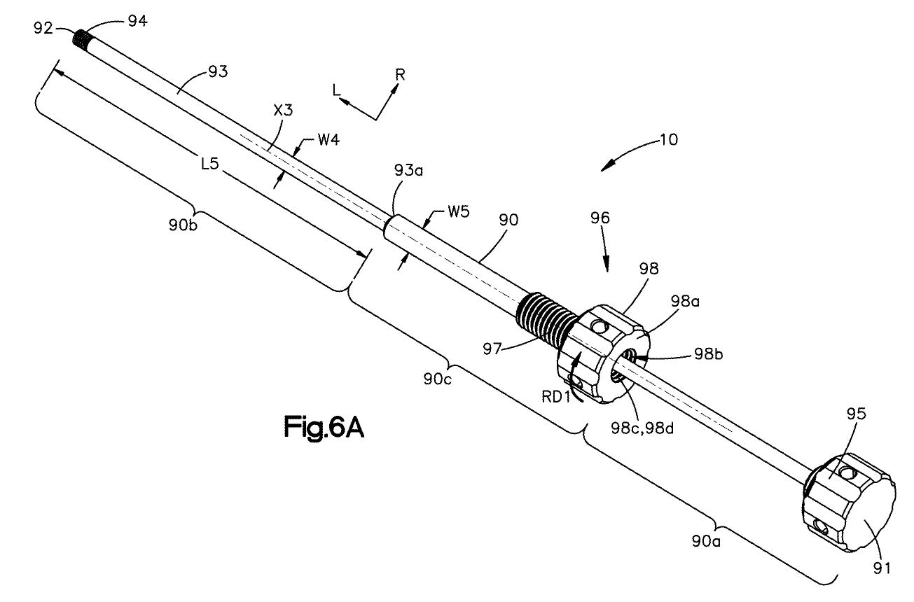

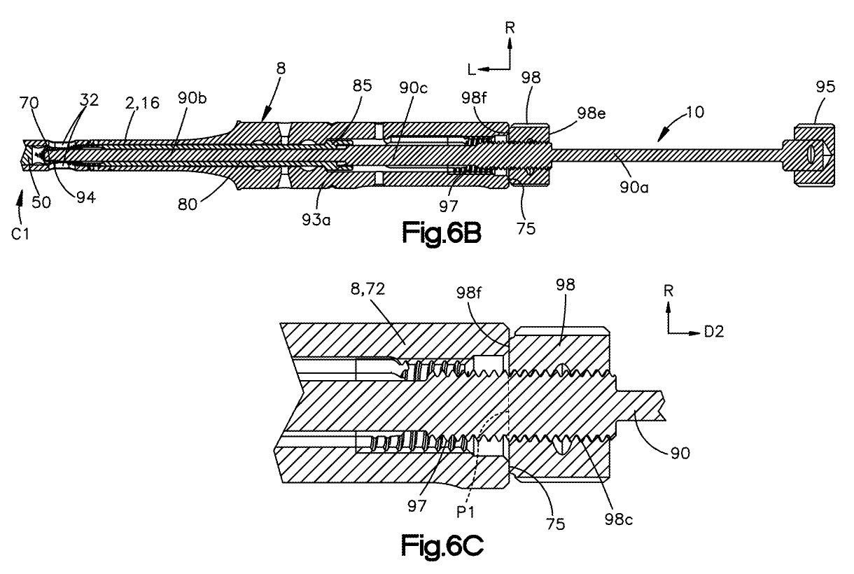

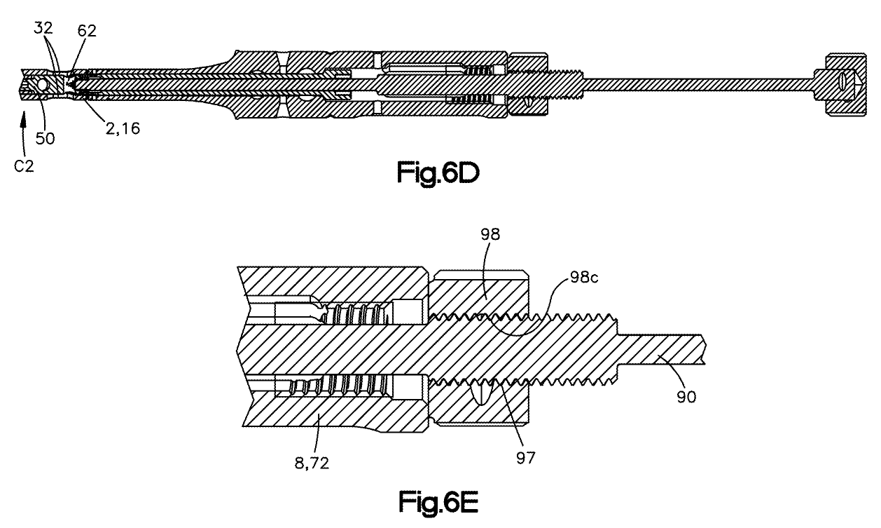

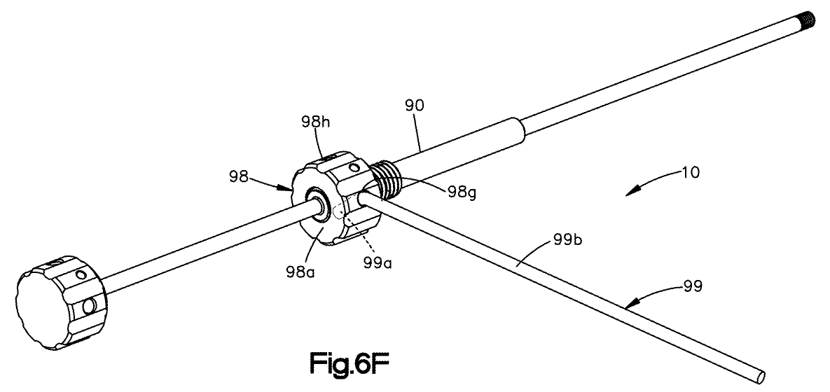

Referring to FIG. 1, an exemplary surgical system 100 is shown for repairing bone. For example, the system 100 can be particularly configured for employing an intramedullary (IM) nail 2 to repair a joint interconnecting a long bone to one or more additional bones of patient anatomy. Accordingly, the surgical system 100 can also be referred to an “IM nailing system” 100. In the illustrated embodiments herein, the system 100 is configured for repairing an ankle joint via a TTC ankle fusion procedure. The system 100 includes an intramedullary (IM) nail 2 and an instrument assembly 4 for implanting the IM nail 2 within bone, such as within a calcaneus 3, talus 5, and tibia 7 for a TTC ankle fusion. The instrument assembly 4 includes an insertion member 8 (which can also be referred to as an “insertion handle” 8 or “nail inserter” 8), and a deployment instrument 10 for deploying an integrated compression mechanism 12 of the IM nail 2, as described in more detail below. The nail inserter 8 and a connecting screw 80 (see FIGS. 5B-5C and 6B and 6D) are configured to couple with the IM nail, 2, after which the nail inserter 8 is configured to drive the IM nail 2 along an insertion trajectory X through bone and into the medullary canal of a longbone (e.g., tibia 7). The insertion trajectory X can be substantially along the anatomical axis of the tibia 7, for example. The system 100 also preferably includes locking members for affixing the IM nail 2 to adjacent bone. As shown, the locking members can include proximal locking members 13 and distal locking members 14 for locking associated portions of the IM nail 2 to bone. The locking members 13, 14 can be bone screws, anchors, blades (e.g., spiral blades), and the like, and can be sized and configured according to the particular bone regions through which they are configured to extend.

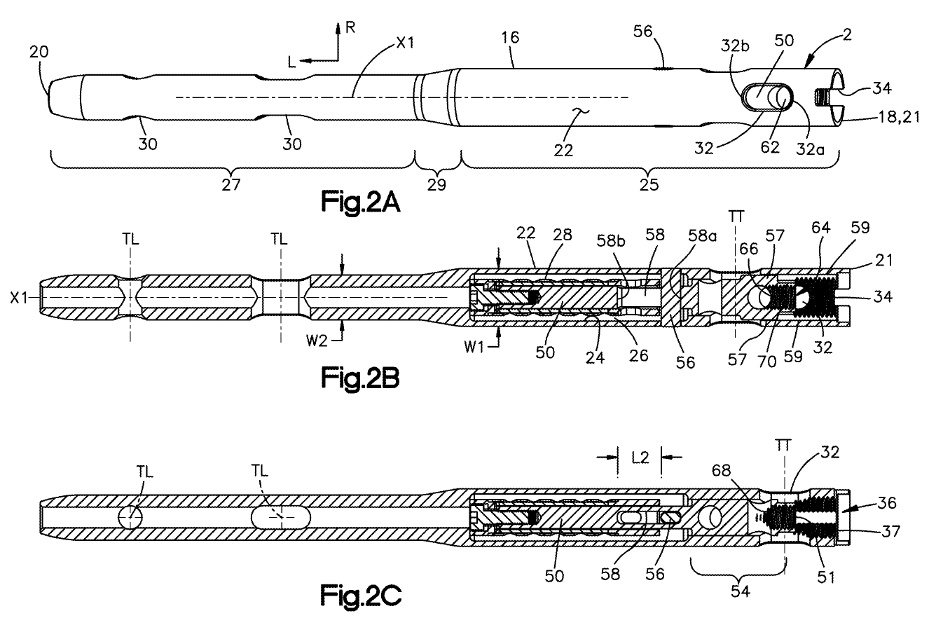

Referring to FIGS. 2A-2C, the IM nail 2 has a nail body 16 that is elongate along a longitudinal nail axis X1. The nail body 16 extends from a trailing nail end 18 to a leading nail end 20 along a longitudinal direction L that is oriented along the longitudinal nail axis X1. In particular, the leading nail end 20 is spaced from the trailing nail end 18 in a forward direction D1, while the nail trailing end 18 is spaced from the nail leading end 20 in a rearward direction D2 opposite the leading direction D1. It should be appreciated that the forward direction D1 and rearward direction D1 are mono-directional components of the longitudinal direction L, which is bi-directional. The nail body 16 has a trailing end surface 21 at the nail trailing end 18. The nail body 16 has an exterior surface 22 and an interior surface 24 spaced from each other along a radial direction R that is perpendicular to the longitudinal nail axis X1. The interior surface 24 defines an interior channel 26 of the nail body 16 that houses the compression mechanism 12, which includes a spring member 28 for providing post-operative bone compression in the bone(s) in which the IM nail 2 is implanted, as described in more detail below. It should be appreciated that, as used herein: the terms “longitudinal”, “longitudinally”, and derivatives thereof refer to the longitudinal direction L; the terms “forward”, “forwardly”, and derivatives thereof refer to the forward direction D1; and the terms “rearward”, “rearwardly”, and derivatives thereof refer to the rearward direction D2; and the term “radially” and derivatives thereof refer to the radial direction R.

The leading nail end 20 is configured to lead insertion of the IM nail 2 into bone and can thus have a tapered leading tip geometry. The trailing nail end 18 is configured to couple with the nail inserter 8, which is configured to be manipulable for controlling movement of the IM nail 2 during nail insertion into bone. The nail body 16 includes a trailing portion 25 that extends from the trailing end 18 toward the leading end 20 and also includes a leading portion 27 that extends from the leading end 20 toward the trailing portion 25. The nail body 16 can also have an intermediate portion 29 positioned longitudinally between the trailing and leading portions 25, 27. As shown, the leading portion 27 can have a cross-sectional dimension W2 that is narrower than a cross-sectional dimension W1 of the trailing portion 25, which can facilitate nail insertion into the medullary canal of the respective receiving bone.

It should be appreciated that, in the field of TTC ankle fusion procedures, IM nails are configured to be inserted along an insertion trajectory X (see FIG. 1) through the calcaneus 3 and talus 5 and into the tibia 7 moving in an anatomical proximal direction. Thus, for use in such procedures, the leading portion 27 and leading end 20 of the IM nail 2 are often referred to as the respective “proximal portion” and “proximal end” of the IM nail 2, with reference to their intended implanted positions being generally at or nearest the proximal region of the bone (e.g., proximal tibia) and generally furthest from the distal regions of bone (e.g., distal tibia, talus, and calcaneus). Similarly, for use in such procedures, the trailing portion 25 and trailing end 18 of the nail 2 are often referred to as the respective “distal portion” and “distal end” of the nail 2, with reference to their intended implanted position being generally at or nearest the distal regions of bone and generally furthest from the proximal region of the bone.

The nail body 16 defines a plurality of locking structures for affixing the IM nail 2 to bone. These locking structures include one or more proximal locking holes 30 extending through the leading portion 27 of the nail body 16. The proximal locking hole(s) 30 are configured to receive the proximal locking members 13 discussed above. The locking structures also include at least one distal locking hole 32 extending through the trailing portion 25 of the nail body 16. In particular, the at least one distal locking hole 32 extends generally radially from the interior channel 26 (i.e., from the interior surface 24) to the exterior surface 22 of the nail body 16. The at least one distal locking hole 32 is configured to receive a respective one of the distal locking members 14 discussed above. As shown, the at least one distal locking hole 32 can be longitudinally elongated to allow the at least one distal locking member 14 extending therethrough to travel in the forward direction D1 relative to the nail body 16 during a compression phase of nail operation. The at least one distal locking hole 32 can therefore be characterized as a distal locking slot 32 extending longitudinally from a trailing hole end 32a to a leading hole end 32b. The proximal and distal locking hole(s) 30, 32 extend through the nail body 16 along respective leading and trailing hole axes TL, TT, which can be perpendicular to the longitudinal nail axis X1, as shown. In other embodiment, one or more an up to each of the proximal and distal locking hole(s) 30, 32 can extend through the nail body 16 along a respective hole axis TL, TT that is offset at an oblique angle from the longitudinal nail axis X1.

The trailing end 18 has one or more features for coupling with the nail inserter 8 and for providing access to interior features of the IM nail 2 through the trailing end 18. For example, as shown in FIGS. 2B-2C, the nail trailing end 18 can define one or more recesses 34 that are proximally spaced from the trailing end surface 21 and are configured to matingly receive one or more complementary protrusions of the nail inserter 8. For example, the nail trailing end 18 can define a pair of radially opposed recesses 34 that are configured to matingly receive a pair of radially opposed protrusions of the nail inserter 8. The nail trailing end 18 can also define a rear opening 36 in communication with the interior channel 26 of the nail body 16 for providing access for the instrument assembly 4 to extend within the interior channel 26. For example, the trailing nail portion 25 can have interior threads 37 defined along the interior surface 24 extending forwardly from the trailing nail end 18. The interior threads 37 can be configured to threadedly engage complementary exterior threads defined by the nail inserter 8 for coupling the nail inserter 8 to the IM nail 2.

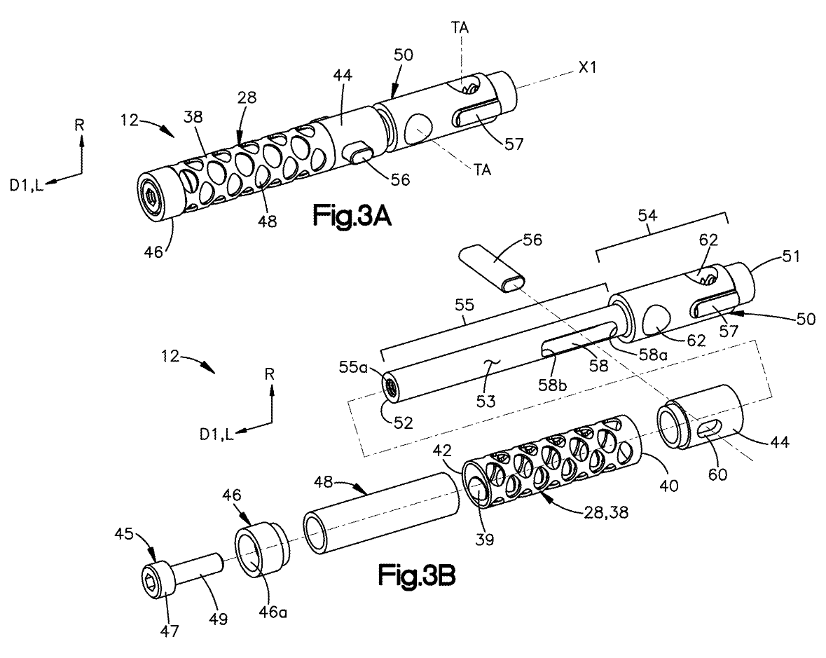

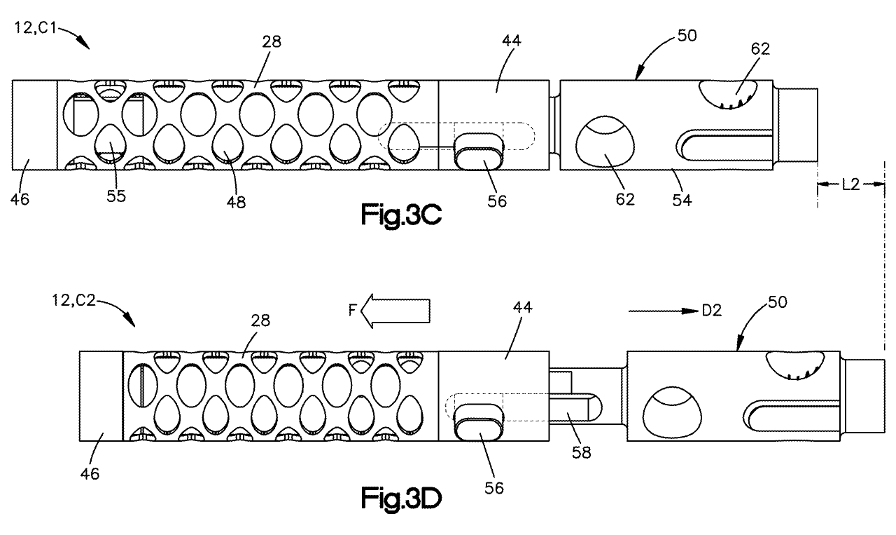

Referring now to FIGS. 3A-3B, the compression mechanism 12 includes a shuttle 50 joined with the spring member 28 within the interior channel 26 in the nail body 16. The spring member 28 has a spring body 38 that provides a spring force for providing the bone compression. The spring body 38 can have various configurations that provide the desired spring force. In the illustrated embodiment, the spring body 38 has a trailing spring end 40 and a leading spring end 42 spaced from each other along the longitudinal direction L. The spring body 38 can also define an interior spring channel 39 that extends longitudinally between the trailing and leading spring ends 40, 42. The compression mechanism 12 can also include features for coupling the spring member 28 with the shuttle 50. For example, in the illustrated embodiment, the compression mechanism 12 includes a trailing coupling member 44 that engages with the trailing spring end 40 and a leading coupling member 46 that engages with the leading spring end 42. The compression mechanism 12 can also include an intermediate coupling member 48 that extends between the trailing and leading coupling members 44, 46 and interfaces with the spring member 28. The trailing, leading, and intermediate coupling members 44, 46, 48 can be configured to interface with respective components of the IM nail 2 to operate the spring member 28 in a manner providing bone compression, as described in more detail below.

The spring member 28 is configured such that, when loaded, the spring force F is exerted in the forward direction D1, which provides the compressive force in the bone(s) affixed to the IM nail 2. In the illustrated embodiments herein, the spring member 28 is a compression-type spring that is compressed longitudinally to load the spring force F in the forward direction D1. However, in other embodiments, the spring member 28 can be a tension-type spring that is subjected to longitudinal tensile force(s) to load the spring force F in the forward direction D1. It should be appreciated that various spring configurations can be employed to provide the compression mechanism 12 with a spring force in the forward direction D1. As shown, the spring body 38 can have a cylindrical, mesh-like geometry comprising a plurality of legs interconnected by a plurality of nodes which facilitate and enhance the elastic deformation properties of the spring body 38. The spring body 38 is preferably constructed of nitinol, a nickel-titanium alloy having super-elastic and shape-memory properties. However, the spring body 38 can optionally be constructed of different materials and can have different geometries and configurations that provide a necessary spring force for bone compression.

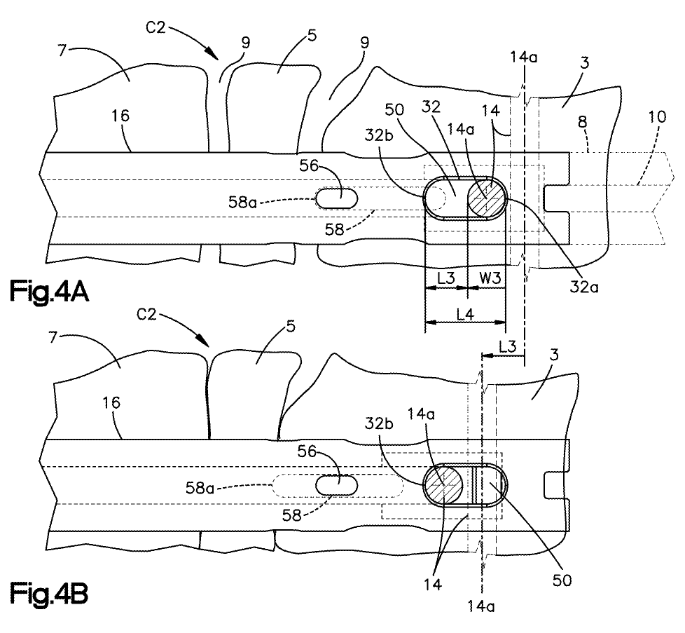

Referring now to FIGS. 3C-3D, the compression mechanism 12 is configured to transition between a first configuration C1 (FIG. 3C), in which the spring member 28 possesses a minimum spring force, and a second configuration C2 (FIG. 3D), in which the spring member 28 is loaded with a maximum spring force F. The spring member 28 is also configured to transition from the second configuration C2 forwardly toward a third configuration C3 (shown in FIG. 4B) in a manner providing bone compression. Accordingly, when the IM nail 2 is proximally and distally locked to bone, transition between the second configuration C2 toward the third configuration C3 can be referred to herein as the “compression phase” of IM nail operation.

The first and second configurations C1, C2 are intra-operative configurations, and transition from the second configuration C2 toward the third configuration C3 can commence intra-operatively and continue post-operatively. In particular, the IM nail 2 is configured to provide post-operative bone compression (from C2 toward C3) to enhance patient outcomes. It should be appreciated that, based on patient factors, the IM nail 2 need not actually achieve the maximum compression provided by the IM nail 2 to provide satisfactory patient outcomes. Thus, reference is made herein to the nail transitioning “toward” the third configuration C3. It should therefore be appreciated that the IM nail 2 need not necessarily transition entirely to the third configuration C3 to achieve the full scope of benefits described herein. It should also be appreciated that, when the IM nail 2 is proximally and distally locked, any measurable transition between the second configuration C2 toward the third configuration C3 can provide bone compression.

The IM nail 2 can be configured such that, in the first configuration C1, the spring member 28 is in a neutral or “at-rest” geometry, such that it is not subjected to any load and therefore possesses no spring force to counteract the load. In other embodiments, the IM nail 2 can be configured such that the spring member 28 can be subjected to a non-zero load when in the first configuration C1. Thus, in such embodiments, the spring member 28 can possess some measure of spring force in the first configuration C1.

The shuttle 50 can be configured to facilitate transition of the spring member 28 from the first configuration C1 to the second configuration C2 and from the second configuration C2 to the third configuration C3. The shuttle 50 can also be configured to receive the distal locking member(s) 14 and transmit the spring force F thereto from the second configuration C2 toward the third configuration C3, thereby providing the post-operative bone compression.

Referring again to FIGS. 3A-3B, the shuttle 50 extends longitudinally from a trailing shuttle end 51 to a leading shuttle end 52 and has an outer surface 53 extending between the trailing and leading shuttle ends 51, 52. In the illustrated embodiment, the spring member 28 is disposed along a leading portion 55 of the shuttle 50 spaced forwardly from a trailing portion 54 of the shuttle 50. The trailing shuttle portion 54 extends in the forward direction D1 from the trailing shuttle end 51 toward the leading shuttle portion 55. The leading shuttle portion 55 extends in the rearward direction D2 from the leading shuttle end 52 toward the trailing shuttle portion 54. As shown, the leading shuttle portion 55 is configured to extend within the interior spring channel 39. Additionally, the intermediate coupling member 48 extends within the interior spring channel 39 and resides annularly between the leading shuttle portion 55 and the spring member 28 in sleeve-like fashion, while the leading shuttle portion 55 extends within a longitudinal channel 48a of the intermediate coupling member 48. The intermediate coupling member 48 can contact the trailing coupling member 44 and/or the leading coupling member 46 during transition from the first configuration C1 to the second configuration C2. Additionally, the leading coupling member 46 can be attached to the leading shuttle portion 55.

In the illustrated embodiment, a fastener 45 attaches the leading coupling member 46 with the leading shuttle portion 55. As shown, the fastener 45 can be a threaded bolt 45 having a head 47 and a shaft 49 that extends longitudinally from the head 47 and defines external threads. The head 47 can be configured to seat within a receptacle 46a of the leading coupling member 46, while the shaft 49 is configured to threadedly engage interior threads defined on an interior surface within a leading bore 55a defined within, and extending longitudinally along, the leading shuttle portion 55. The leading bore 55a extends in the rearward direction D2 from the leading shuttle end 52 toward the trailing shuttle end 51. The bore 55a is preferably a blind bore, although in other embodiments the bore 55a can extend continuously to the trailing shuttle end 51. It should be appreciated that the spring member 28 can be coupled with the shuttle 50 using various coupling mechanisms in addition to, or as an alternative to, the fastener 45 while remaining within the scope of the present disclosure.

The trailing shuttle portion 54 has engagement formations for coupling with the deployment instrument 10 (FIG. 1) in a manner allowing the deployment instrument 10 to transition the shuttle 50 from the first configuration C1 to the second configuration C2. For example, as shown in FIGS. 2B-2C, the trailing shuttle portion 54 can define a rear opening 64 in communication with a bore 66 extending forwardly within the shuttle 50 from the trailing shuttle end 51. An interior surface 68 of the shuttle 50 within the bore 66 can define internal threads 70, which can be configured to threadedly engage exterior threads of the deployment instrument 10. It should be appreciated that other engagement formations configurations can be employed for facilitating coupling of the deployment instrument 10 to the shuttle 50.

Referring now to FIGS. 2A-2B and 3C-3D, the IM nail 2 is configured to provide controlled, guided movement of the shuttle 50 and the spring member 28 between the first, second, and third configurations C1, C2, C3. In the illustrated embodiment, the shuttle 50 is configured to be retracted rearwardly in a manner such that the trailing spring end 40 abuts a stop member of the IM nail 2 in a manner longitudinally compressing the spring member 28, thereby loading the spring force F, during transition from the first configuration C1 to the second configuration C2, as shown in FIGS. 3C-3D. The stop member can be the trailing coupling member 44, as shown, which can be coupled to a pin 56 extending transversely through the nail body 16 to maintain the position of the stop member (i.e., the trailing coupling member 44) relative to the nail body 16. The pin 56 also extends through a pair of opposed slots 60 extending transversely through the trailing coupling member 44 (one of the slots 60 is shown in FIG. 3B). The pin 56 can also extend transversely through a longitudinal slot 58 defined by the shuttle 50, which allows the shuttle 50 to translate longitudinally relative to the pin 56, as described in more detail below.

The slot 58 can extend longitudinally between a trailing slot end 58a and a leading slot end 58b. The slot 58 defines a longitudinal length between the trailing and leading slots ends 58a,b that is greater than a longitudinal dimension of the pin 56 so as to define a maximum travel length L2 of the shuttle 50 relative to the pin 56. In the illustrated embodiment, the maximum travel length L2 also defines the maximum longitudinal distance by which the spring member 28 can be elastically deformed (e.g., compressed) moving from the first configuration C1 to the second configuration C2 to load the spring force F. In the illustrated embodiment, the IM nail 2 is configured such that some measure of longitudinal clearance is present between the pin 56 and the trailing and leading slot ends 58a,b in the first and second configurations C1, C2, respectively, as shown in FIG. 3C. In other embodiments, however, the IM nail 2 can be configured such that the pin 56 abuts the trailing slot end 58a when the spring member 28 is in the first configuration C1, and/or the pin 56 abuts the leading slot end 58b when the spring member 28 is in the second configuration C1. In such other embodiments, the pin 56 and the slot 58 can be cooperatively configured such that interaction therebetween can provide a hard stop when the shuttle 50 has translated to the second configuration C2, thereby indicating to the user that the compression mechanism 12 has transitioned to the second configuration C2.

The shuttle 50 and the nail body 16 can have one or more complementary features for guiding movement of the shuttle 50 relative to the nail body 16. In the illustrated embodiment, the slot 58 and the pin 56 can be cooperatively configured to prevent shuttle 50 rotation about the longitudinal nail axis X1 as the shuttle 50 translates between the first and second configurations C1, C2. Additionally, the shuttle 50 can have one or more protrusions 57 or “runners” 57 that extend radially outward from the outer shuttle surface 53 and are configured to ride along complementary structures, such as guide slots 59 of the nail body 16. For example, the shuttle 50 can have a pair of opposed runners 57 that extend within, and ride along, a pair of complementary guide slots 59 defined by the trailing portion 25 of the nail body 16 (see FIG. 2B), in a manner preventing shuttle rotation 50 relative to the nail body 16 about the longitudinal nail axis X1. It should be appreciated that other complementary guide features for guiding movement of the shuttle 50 relative to the nail body 16 are within the scope of the present disclosure.

The shuttle 50 defines at least one distal aperture 62 configured to receive at least one distal locking member 14 for affixing the shuttle 50 to bone. The locking mechanism 12 is configured such that the at least one distal aperture 62 of the shuttle 50 is in alignment with the at least one distal locking hole 32 when in the second and third configurations C2, C3 (see FIGS. 4A-4B), and optionally when in the first configuration C1. In this manner, to lock the IM nail 2 within bone distally, a distal locking member 14 can be inserted along an insertion trajectory sequentially through bone on the near side of the IM nail 2, through the near portion of the distal locking hole 32, through the distal aperture 62, through the far portion of the distal locking hole 32, and into bone on the far side of the IM nail 2. As shown, the at least one distal aperture 62 can include two (2) distal apertures 62 that are longitudinally spaced from each other.

Each distal aperture 62 extends through the shuttle 50 along an aperture axis TA angularly offset from the longitudinal nail axis X1. As shown, the aperture axes TA of the two distal apertures 62 can each be perpendicular to the longitudinal nail axis X1 and can be angularly offset from each other at about 90 degrees about the longitudinal nail axis X1. In other embodiments, one or more of the distal apertures 62 can be oriented at an oblique angle to the longitudinal nail axis X1. In additional embodiments, the distal apertures 62 can be parallel with each other and can both be aligned with and translate along the same pair of radially opposed distal locking holes 32 of the nail body 16. In further embodiments, the distal apertures 62 can be angularly offset from each other at an oblique angle about the longitudinal nail axis X1. In yet other embodiments, the shuttle 50 can define three (3) or more distal apertures 62, which can be oriented relative to one another at any of the various orientations described above.

Referring now to FIGS. 4A-4B, operation of the compression mechanism 12 of the IM nail 2 will now be described. As shown in FIG. 4A, with the nail inserter 8 coupled to the IM nail 2, and with the deployment instrument 10 retaining the shuttle 50 (and the spring member 28) in the second configuration C2, distal locking can be performed on the IM nail 2. It should be appreciated that, in the IM nailing system 100 described herein, distal locking of the IM nail 2 is preferably performed before proximal nail locking. However, the IM nail 2 is configured such that proximal locking can optionally be performed before distal locking. During distal nail locking, one or more distal locking member 14 can be inserted through bone and through one or more associated distal locking holes 32 of the nail body 16 and aligned distal apertures 62 of the shuttle 50. In the illustrated example, a pair of distal locking members 14 are both inserted through the calcaneus 3 along perpendicular locking member axes 14a that intersect an associated pair of distal locking holes 32 and distal apertures 62. After distal locking and proximal locking have been completed, the deployment instrument 10 can be uncoupled from or otherwise release the shuttle 50.

As shown in FIG. 4B, after the shuttle 50 is released from the deployment instrument 10, the spring force F of the loaded spring member 28 applies compressive force between the bone material locked to the trailing nail portion 25 (e.g., the calcaneus 3) and the bone material locked to the leading nail portion 27 (e.g., the tibia 7). In the illustrated example, the shuttle 50 and the spring member 28 are configured to drive the distal locking members 14 and the calcaneus 3 locked therewith in the forward direction D1 (i.e., in the anatomical proximal direction) toward the tibia 7. It should be appreciated that the spring member 28 can also pull the proximally locked bone material (e.g., the tibia 7) toward the distally locked bone material (e.g., the calcaneus 3). The bone compression provided by the IM nail 2 can reduce inter-bone gaps 9 between bone segments locked to and/or adjacent to the nail 2. For example, with reference to a TTC ankle fusion procedure, the compression provided by the IM nail 2 can thereby reduce inter-bone gaps 9 between the calcaneus 3, talus 5, and tibia 9.

The IM nail 2 defines a maximum compression distance L3 from the second configuration C2 to the third configuration C3. In the illustrated embodiment, the maximum compression distance L3 is a function of the outer diameter W3 of the shaft of the distal locking member 14 extending through the distal locking hole 32, and a longitudinal hole length L4 of the distal locking hole 32. In particular, the IM nail 2 can be configured such that, in the second configuration C2, the distal aperture 62 of the shuttle 50 is longitudinally positioned relative to the nail body 16 such that the distal aperture 62 and the trailing end 32b of the distal locking hole 32 cooperatively define a substantially circular opening through the nail body 16 to receive the shaft of the distal locking member 14. This alignment can be controlled by the leading end 58b of the longitudinal shuttle slot 58 abutting the pin 56 rearwardly, as described above.

After the distal locking member 14 is inserted through the distal locking hole 32 and distal aperture 62, and during the compression phase, the shaft of the distal locking member 14 can travel forwardly relative to the nail body 16 along the distal locking hole 32 until the shaft abuts the leading end 32b of the distal locking hole 32. Accordingly, the maximum compression distance L3 of the illustrated embodiment is the difference between the longitudinal hole length L4 and the outer shaft diameter W3 (i.e., L3 =L4–W3). It should be appreciated that the IM nail 2 can be configured such that other factors determine the maximum compression distance L3, such as interaction between the trailing end 58a of the longitudinal shuttle slot 58 and the pin 56, by way of a non-limiting example.

With reference to FIGS. 5A-6F, the instrument assembly 4 of the IM nailing system 100 will now be more fully described. In particular, the nail inserter 8 will be more fully described with reference to FIGS. 5A-5C; and the deployment instrument 10 will be more fully described with reference to FIGS. 6A-6F.

Referring now to FIGS. 5A-5C, the nail inserter 8 has an inserter body 72 that is elongate along a longitudinal inserter axis X2 that is configured to be parallel with the longitudinal nail axis X1 when the inserter 8 is coupled to the IM nail 2. Accordingly, the longitudinal inserter axis X2 is oriented along the longitudinal direction L when the nail inserter 8 is coupled to the IM nail 2. Preferably, the nail inserter 8 is configured such that the longitudinal inserter axis X2 is coaxial with the longitudinal nail axis X1 when the nail inserter 8 is coupled with the IM nail 2. For purposes of discussion, the spatial relationships between various features of the nail inserter 8 and other features of the system 100 will be described with reference to the longitudinal, forward, rearward, and radial directions L, D1, D2, R described above, with the understanding that such directional terms refer to such features of the nail inserter 8 in its orientation when coupled to the IM nail 2.

The inserter body 72 extends from a trailing inserter end 74 to a leading inserter end 76 spaced from each other along the longitudinal direction L. The inserter body 72 is configured to be gripped manually for manipulating the IM nail 2 during the implantation procedure. The inserter body 72 has a trailing end surface 75 at the trailing inserter end 74 and a leading surface 77 at the leading inserter end 76. The leading inserter end 76 is configured to couple with the trailing nail end 18 of the IM nail 2, as described in more detail below. The inserter body 72 defines a guide channel, such as a cannulation 78, extending from the trailing inserter end 74 to the leading inserter end 74 in the forward direction D1. The cannulation 78 (also referred to herein as the “inserter cannulation” 78) is preferably configured such that the longitudinal inserter axis X2 extends centrally therethrough. The inserter cannulation 78 is configured to allow one or more instruments, including the deployment instrument 10, to extend therethrough and engage various features of the IM nail 2, such as the shuttle 50, as described in more detail below.

The nail inserter 8 can be configured to couple with the IM nail 2 such that the leading surface 77 of the nail inserter 8 abuts or otherwise interfaces with the trailing end surface 21 of the nail 2. To rigidly couple the nail inserter 8 to the IM nail 2 in such position, the nail inserter 8 can include a coupling member, such as a cannulated connecting screw 80, that extends within the inserter cannulation 78 and is extendable forwardly from the leading inserter end 76. The connecting screw 80 has external threads 82 that are configured to extend with the rear opening 36 of the nail body 16 and threadedly engage the interior threads 37 of the trailing nail portion 25. In this manner, the cannulated connecting screw 80 can be driven into secure threaded engagement with the IM nail 2 in a manner rigidly coupling the nail inserter 8 with the IM nail 2 along the longitudinal direction L. The cannulated connecting screw 80 also has a rear drive coupling, such as a drive socket 84, for receiving a complementary drive bit of the driving tool to drive the threaded engagement between the connecting screw 80 and the IM nail 2. As mentioned above, the connecting screw 80 is cannulated, and thereby defines a screw cannulation 86, extending therethrough along the longitudinal inserter axis X2. Preferably, the screw cannulation 86 is configured such that longitudinal inserter axis X2 extends centrally therethrough.

The nail inserter 8 can have an additional coupling feature, such as a pair of radially opposed protrusions or prongs 88, the extend forwardly from the leading surface 77 of the nail inserter 8. The prongs 88 can be configured to insert within the radially opposed recesses 34 of the trailing nail portion 25. Engagement between the prongs 88 and recesses 34 prevents relative rotation between the nail inserter 8 and the IM nail 2 about the longitudinal nail axis X1. This engagement maintains the orientation of the proximal and distal locking holes 13, 14 with respect to the nail inserter 8, which facilitates hole targeting for proximal and distal locking.

Referring now to FIGS. 6A-6E, the deployment instrument 10 is configured to engage the shuttle 50 and move the shuttle 50 relative to the nail body 16 to transition the compression mechanism 12 (e.g., the shuttle 50 and spring member 28) from the first configuration C1 to the second configuration C2. The deployment instrument 10 is also configured to maintain or “hold” the shuttle 50 in the second configuration C2 during proximal locking of the IM nail 2, and to subsequently disengage the shuttle 50 to commence the compression phase (i.e., transition from the second configuration C2 toward the third configuration C3). As shown in FIG. 6A, the deployment instrument 10 has an elongate instrument body 90 extending along a central instrument axis X3. The instrument body 90 extends longitudinally between a trailing instrument end 91 and an opposed leading instrument end 92. An outer surface 93 of the instrument body 90 extends from the trailing instrument end 91 to the leading instrument end 92.

The instrument body 90 has a trailing instrument portion 90a, a leading instrument portion 90b spaced forwardly from the trailing portion 90a, and an intermediate instrument portion 90c located longitudinally between the trailing and leading instrument portions 90a,b. The leading instrument portion 90b extends forwardly from the intermediate instrument portion 90c to the leading instrument end 92. The trailing instrument portion 90a extends rearwardly from the intermediate instrument portion 90c to the trailing instrument end 91. As shown in FIG. 6B, the leading instrument portion 90b is configured to be inserted through the inserter cannulation 78 (and through the screw cannulation 86), through the rear opening 36 at the trailing nail end 18 (see FIG. 2B), and into engagement with the shuttle 50. Accordingly, the deployment instrument 10 is configured such that the central instrument axis X3 is coaxial with the longitudinal inserter axis X2, which are preferably coaxial with the longitudinal nail axis X2 when the nail inserter 8 and the deployment instrument 10 are coupled with the IM nail 2. As shown, the leading instrument portion 90b can have a cross-sectional dimension W4 that is narrower than a cross-sectional dimension W5 of the intermediate instrument portion 90c. The instrument body 90 can also define a shoulder 93a at the boundary between the leading and intermediate instrument portions 90a,b. The leading instrument portion 90b defines a longitudinal length L5, measured from the shoulder 93a to the leading instrument end 92. The shoulder 93a can optionally facilitate instrument seating within the inserter cannulation 78 (and within the screw cannulation 86).

The leading instrument portion 90b defines engagement structures 94, which can extend to the leading instrument end 92, and are configured engage the complementary engagement formations of the trailing portion 54 of the shuttle 50. The deployment instrument 10 includes a first force actuator 95 configured to move the instrument body 90 relative to the IM nail 2 to cause the engagement structures 94 of the instrument body 90 to engage the complementary engagement formations of the shuttle, thereby attaching the deployment instrument 10 to the shuttle 50 while the shuttle 50 is in the first configuration C1. In the illustrated embodiment, the engagement structures 94 of the leading instrument portion 90b are external threads 94 defined on the exterior surface 93 of the instrument body 90. The external threads 94 can extend to the leading instrument end 92. The complementary engagement features of the shuttle 50 in the illustrated embodiment are the internal threads 70 defined in the bore 66 of the trailing shuttle portion 54, as described above with reference to FIGS. 2B-2C.

In the illustrated embodiment, the first force actuator 95 is a knob 95 disposed along the trailing instrument portion 90a. As shown, the knob 95 (which can also be referred to herein as the “coupling knob” 95) can be positioned at the trailing instrument end 91 and can therefore define the trailing instrument end 91. The knob 95 is configured to be manually manipulated, such as for inserting the leading instrument portion 90b through the inserter cannulation 78 and advancing (e.g., pushing) the leading instrument end 92 forwardly through the rear opening 64 and into the bore 66 of the shuttle 50. In this manner, the knob 95 can facilitate advancing the external threads 94 of the leading instrument portion 90b into position adjacent the internal threads 70 of the trailing shuttle portion 54. The knob 95 is also configured to facilitate manual rotation of the instrument body 90 about the central instrument axis X3, thereby threadedly engaging the external threads 94 of the leading instrument portion 90b with the internal threads 70 of the trailing shuttle portion 54. Thus, the knob 95 can help facilitate coupling the deployment instrument 10 to the shuttle 50 of the IM nail 2.

As shown in FIGS. 6B-6E, with the deployment instrument 10 coupled to the shuttle 50, the deployment instrument 10 can be employed to retract the shuttle 50 rearwardly into the second configuration C2. The deployment instrument 10 includes a translation mechanism 96 for providing controlled, rearward translation of the instrument body 90 (and the shuttle 50 coupled thereto) relative to the nail body 16 to the second configuration C2. The translation mechanism 96 includes one or more transmission structures 97 defined on the intermediate instrument portion 90c, and also includes a second force actuator 98 that is connectable to the intermediate instrument portion 90c. The second force actuator 98 defines one or more complementary transmission formations 98c configured to engage the one or more transmission structures 97 in a manner causing the instrument body 90 to translate rearwardly relative to second force actuator 98.

In the illustrated embodiment, the one or more transmission structures 97 include external transmission threads 97 defined on the exterior surface 93 of the intermediate instrument portion 90c; the second force actuator 98 is a second knob 98 (also referred to herein as the “transmission knob” 98) having a knob body 98a; and the one or more complementary transmission formations 98c include internal transmission threads 98c defined on an interior surface 98d within a central bore 98b of the transmission knob body 98a. The central bore 98b extends through the transmission knob 98 from a trailing end surface 98e to a leading end surface 98f of the transmission knob 98. The internal transmission threads 98c are configured to threadedly engage the external transmission threads 97 in a manner to convert rotational motion of the transmission knob 98 into translational motion of the instrument body 90. This rotational-to-translational motion is employed to retract the shuttle 50 rearwardly relative to the nail body 16 to transition the compression mechanism 12 to the second configuration C2, as described in more detail below. The transmission knob 98 can be slidably coupled to the instrument body 90 such that, when not threadedly engaged with the external transmission threads 97, the transmission knob 98 can slide freely along the trailing instrument portion 90a between the coupling knob 95 and the external transmission threads 97.

As shown in FIGS. 6B-6C, the leading instrument portion 90b and at least some of the intermediate instrument portion 90c are insertable within the inserter cannulation 78. The length L5 of the leading instrument portion 90b is sized so that, when the deployment instrument 10 is fully seated within the inserter cannulation 78, as shown in FIG. 6B, the external threads 94 at the leading instrument end 92 can be fully threadedly engage with the internal threads 70 of the shuttle 50. As shown in FIG. 6C, when the deployment instrument 10 is fully seated within the inserter cannulation 78, some of the external transmission threads 97 are positioned rearward of the trailing end surface 75 of the inserter body 72. This allows the transmission knob 98 to threadedly engage the external transmission threads 97 and to abut the trailing end surface 75 of the inserter body 72 when the deployment instrument 10 is seated within the inserter cannulation 78.

As shown in FIG. 6C, a radial reference line indicates a reference start position P1 of the transmission knob 98, particularly of the leading surface 98f thereof, at which the leading surface 98f can be positioned longitudinally along the external transmission threads 97 without interfering with the threaded coupling of the deployment instrument 10 to the shuttle 50. Thus, when coupling the deployment instrument 10 with the shuttle 50, it is preferable that the leading surface 98f of the transmission knob 98 be located at or rearward of the reference start position P1 shown in FIG. 6C. If necessary, to ensure the transmission knob 98 is positioned at or rearward of the reference start position P1, the user can rotate the transmission knob 98 to translate it rearwardly. It should be appreciated, however, that even if the transmission knob 98 extends forwardly over the reference start position P1 during instrument 10 to shuttle 50 coupling, the coupling knob 95 can nonetheless be employed to couple the instrument 10 with the shuttle 50 (i.e., to fully threadedly engage the external threads 94 of the instrument 10 with the internal threads 70 of the shuttle 50). In particular, rotating the coupling knob 95 rotates the instrument body 90, which will advance the instrument body 90 forwardly relative to the transmission knob 98 by virtue of the threaded engagement of the internal threads 98c of the transmission knob 98 with the external transmission threads 97 of the instrument body 90.

With the deployment instrument 10 seated within the inserter cannulation 78 and coupled to the shuttle 50, and with the leading surface 98f of the transmission knob 98 abutting the trailing end surface 75 of the inserter body 72, as shown in FIG. 6B, the deployment instrument 10 can be employed to deploy the compression mechanism 12 of the IM nail 2. In particular, from this position, the transmission knob 98 can be manually rotated in a first rotational direction RD1 (e.g., clockwise) about the central instrument axis X3 to thereby drive the instrument body 90, and the shuttle 50 coupled thereto, in unison in the rearward direction D2 relative to the nail body 16. This rearward translation moves the shuttle 50 from the first configuration C1 to the second configuration C2, as shown in FIG. 6D, which displaces the spring member 28 and thereby loads the spring force F in the forward direction D1. The rearward translation also moves the distal aperture(s) 62 of the shuttle 50 into desired alignment with the associated distal locking hole(s) 32.

The nail inserter 8 and deployment instrument 10 are configured such that, when in the second configuration C2, the nail inserter 8 and deployment instrument 10 maintain (e.g., hold) the shuttle 50 in the second configuration C2 without further manipulation of the deployment instrument 10 by the user. As shown in FIG. 6E, the deployment instrument 10 can be configured so that the transmission knob 98 remains substantially fully threadedly engaged with the external transmission threads 97 when in the second configuration C2, which facilitates the deployment instrument 10 maintaining the shuttle 50 in the second configuration C2. It should be appreciated that in the second configuration C2, the shuttle 50 preferably abuts at least one of the nail inserter 8 and the cannulated connecting screw 80 in a manner providing a hard stop, which can indicate to the user that the IM nail 2 has transitioned to the second configuration C2.

Referring now to FIG. 6F, the deployment instrument 10 can include one or more structures 99 to facilitate operation of the transmission knob 98. For example, one such structure 99 can be a rotational force multiplier 99, such as a wrench member, such as a pin wrench 99, that is attachable to the transmission knob 98 and configured to be manually manipulated to increase the torque applied to the transmission knob 98. As shown, the pin wrench 98 has a first portion 99a for attachment to the knob 98, such as via insertion within a hole 98g extending radially into the knob body 98a from an exterior surface 98h of the knob body 98a. It should be appreciated that other types of rotational force multipliers can be employed with the transmission knob 98.

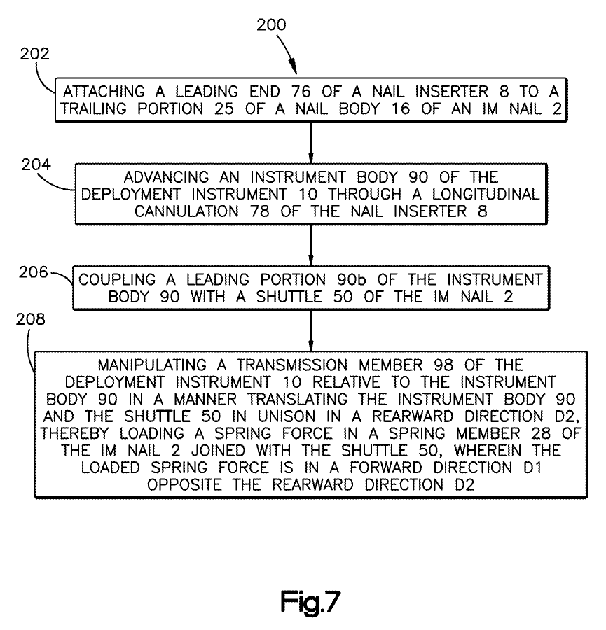

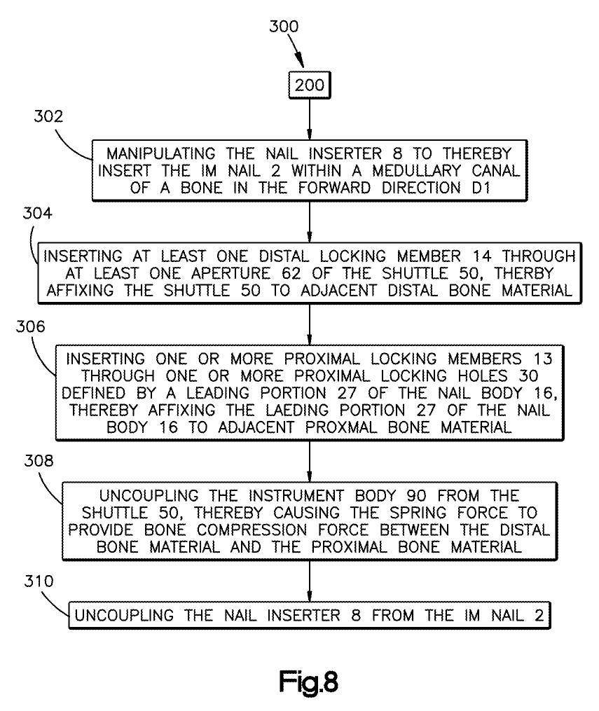

Exemplary methods relating to the IM nailing system 100 will be described with reference to FIGS. 7 and 8.

Referring now to FIG. 7, an exemplary method 200 is described that employs the IM nailing system 100 to prepare the IM nail 2 for implantation in a manner to provide bone compression, such as for a TTC ankle fusion procedure, by way of a non-limiting example. At step 202, a user attaches the nail inserter 8 to the IM nail 2. In particular, the user attaches the leading end 76 of the nail inserter 8 to the trailing nail portion 25 of the nail body 16. During this step, the user inserts the radially opposed prongs 88 of the nail inserter 8 into the recesses 34 at the trailing nail portion 25 and causes the leading surface 77 of the nail inserter 8 to abut the trailing nail surface 21 of the nail body 16. Additionally, the user drives the cannulated connecting screw 80 to threadedly engage the external screw threads 82 with the interior threads 37 of the trailing nail portion 25 (see FIG. 5C). With the prongs 88 received in the recesses 34 and the external screw threads 82 engaged with the interior threads 37, the IM nail 2 is rotationally and longitudinally affixed to the nail inserter 8.

At step 204, the instrument body 90 of the deployment instrument 10 is inserted through the inserter cannulation 78 and into engagement with the shuttle 50 of the IM nail 2. During this step, the user advances the leading portion 90b of the instrument body 90 forwardly through the inserter cannulation 78 and through the screw cannulation 86 so that the leading instrument end 92 extends through the rear opening 36 of the nail body 16. The deployment instrument 10 is further advanced into the bore 66 of the shuttle 50 until the external threads 94 of the leading instrument portion 90b contact, or are immediately adjacent, the internal threads 70 of the trailing shuttle portion 54.

From the position at the conclusion of step 204, the user performs step 206, which includes coupling the leading portion 90b of the instrument body 90 with the shuttle 50. During step 206, the user manipulates the coupling knob 95 to rotate the instrument body 90 about the central instrument axis X3, while applying forward pressure, to cause the external threads 94 of the instrument 10 to threadedly engage the internal threads 70 of the shuttle 50. Preferably, the coupling knob 95 is rotated until the external threads 94 are fully threadedly engaged with the internal threads 70. At the conclusion of step 206, the deployment instrument 10 is coupled to the shuttle 50, preferably while the shuttle 50 remains in the first configuration C1. Preferably, the leading surface 98f of the transmission knob 98 is either coincident with or rearward of the reference start position P1 during step 206.

At step 208, the deployment instrument 10 is translated rearwardly to transition the compression mechanism 12 of the IM nail 2 to the second configuration C2. During this step, the user manipulates the transmission knob 98 by rotating it in the first rotational direction relative to the instrument body 90. This causes the internal transmission threads 98c of the transmission knob 98 to threadedly engage the external transmission threads 97 of the instrument body 90 in a manner translating the instrument body 90, and the shuttle 50 coupled thereto, in unison in the rearward direction D2, thereby elastically deforming the spring member 28 to load the spring force F in the forward direction D1. The transmission knob 98 can be rotated until the leading slot end 58b of the shuttle 50 abuts the pin 56, which occurs when the compression mechanism 12 is in the third configuration C3. Optionally, for patient anatomy that requires less than the maximum extent of bone compression provided by the IM nail 2, the transmission knob 98 can be rotated to translate the shuttle 50 rearwardly to a configuration intermediate the second and third configurations C2, C3.

At the conclusion of step 208, the IM nail 2 is prepared for implantation with the spring force loaded. It should be appreciated that in alternative methods the IM nail 2 can optionally be inserted into patient anatomy before the spring force is loaded.

Referring now to FIG. 8, an exemplary method 300 is described for implanting the IM nail 2 during a TTC ankle fusion procedure. In this exemplary method 300, the IM nail 2 has been prepared for implantation via method 200 described above. Accordingly, method 200 is shown as an initial step in the method 300 for implanting the IM nail 2. At step 302, the IM nail 2 is inserted into patient anatomy. In particular during step 302, the nail inserter 8 is manually manipulated to insert the IM nail 2 along an insertion trajectory X through the calcaneus 3, talus 5, and into the medullary canal of the tibia 7 (see FIG. 1) until the IM nail 2 is located at a desired position with respect to the patient anatomy. Step 302 can include driving the IM nail 2 through the patient anatomy to the desired position, such as by impacting the nail inserter 8 with an impaction hammer. It should be appreciated that the insertion trajectory X can be prepared before step 302, such as by pre-drilling a pilot borehole through the bones 3, 5, 7 in a direction substantially along the anatomical axis of the tibia 7. Additional preparation steps can include inserting a guide wire along the pilot borehole and driving a leading tip of the guidewire into bone (e.g., tibia 7) at the leading end of the borehole, and advancing one or more surgical tools, such as a reamer, to increase the diameter of the borehole. Further preparatory steps can also be employed before inserting the IM nail 2 into the patient anatomy.

At step 304, with the IM nail 2 at the desired position in patient anatomy, and with the shuttle 50 retracted to the second configuration C2 (or optionally to a position intermediate the second and third configurations C2, C3), distal locking of the IM nail 2 is performed. During this step, the one or more distal locking members 14 are inserted through the one or more distal locking holes 32 of the nail body 16 and through the one or more associated distal apertures 62 of the shuttle 50, thereby locking the trailing portion 25 of the IM nail 2 to bone. In particular, for the exemplary TTC ankle fusion procedure, the one or more distal locking members 14 can each be inserted along a locking member trajectory (i.e., along the locking member axis 14a) that extends sequentially through: the near portion of the calcaneus 3; the near portion of the distal locking hole 32; the distal aperture 62; the far portion of the distal locking hole 32; and into the far portion of the calcaneus 3. In the illustrated embodiment, a first distal locking member 14 can be inserted along an insertion trajectory 14a generally along one of the posterior and anterior directions of patient anatomy, and a second distal locking member 14 can be inserted along an insertion trajectory 14a generally along the other of the posterior and anterior directions of patient anatomy. It should be appreciated that other insertion trajectories for one or both of the first and second distal locking members 14 can be employed.

At step 306, proximal locking of the IM nail 2 is performed. During this step, the one or more proximal locking members are inserted through the one or more proximal locking holes 13, thereby locking the leading portion 27 of the IM nail 2 to the tibia 7.

At step 308, with the IM nail 2 distally and proximally locked to patient anatomy, the deployment instrument 10 can be uncoupled from the shuttle 50. This is preferably performed by rotating the coupling knob 95 in the opposite rotational direction employed in step 208, thereby causing the external threads 94 of the instrument 10 to threadedly disengage the internal threads 70 of the shuttle 50. Once threaded disengagement occurs, the coupling knob 95 can be pulled rearwardly to withdraw the deployment instrument 10 from the IM nail 2 and from the nail inserter 8.

At additional step 310, the nail inserter 8 is uncoupled from the IM nail 2. This step includes threadedly disengaging the external threads 82 of the cannulated connecting screw 80 from the internal threads 37 of the trailing portion 25 of the nail body 16, and withdrawing the nail inserter 8 rearwardly away from the IM nail 2.

It should be appreciated that the foregoing methods 200, 300 can include additional and/or alternative steps while remaining within the scope of the present disclosure. It should also be appreciated that the sequence of various steps in the foregoing methods 200, 300 can be adjusted as needed.

As mentioned above, the spring member 28 can have other configurations than the exemplary configuration shown in FIGS. 2B-2C and 3A-3B. For example, the spring member 28 can be a compression coil spring, which can be loaded in a manner generally similar to that shown in the Figures. In other embodiments, the spring member 28 can be a tension-type spring, such as a tension coil spring, that is loaded in response to a tensile force applied by translating the shuttle 50 rearwardly. In such embodiments, the tension spring member can be rigidly coupled to the nail body 16 at a leading portion of the spring. In embodiments involving a coil spring member, the coil spring member can have a diameter that varies along the length of the spring in a manner to provide a favorable loaded spring force F in response to the shuttle 50 translation. It should be appreciated that various additional or alternative spring configurations can be employed to provide the compression mechanism 12 with a spring force F in the forward direction.

Additionally, in other embodiments, the second force actuator 98 of the deployment instrument 10 can have alternative configurations to that shown in the illustrated embodiment. For example, instead of a rotatable knob 98 that threadedly engages with transmission threads of the instrument body 90, the second force actuator 98 can employ a lever-type mechanism, such as a clamp, such as a Destaco-type clamp device that can be squeezed to translate the instrument body 90 (and the shuttle 50 coupled thereto) in the rearward direction D2. It should be appreciated that various additional or alternative force actuators 98 can be employed to translate the instrument body 90 (and the shuttle 50) rearwardly.

Although the disclosure has been described in detail, it should be understood that various changes, substitutions, and alterations can be made herein without departing from the spirit and scope of the invention as defined by the appended claims. Moreover, the scope of the present disclosure is not intended to be limited to the particular embodiments described in the specification. In particular, one or more of the features from the foregoing embodiments can be employed in other embodiments herein. As one of ordinary skill in the art will readily appreciate from that processes, machines, manufacture, composition of matter, means, methods, or steps, presently existing or later to be developed that perform substantially the same function or achieve substantially the same result as the corresponding embodiments described herein may be utilized according to the present disclosure.

What is claimed:

1. An instrument assembly for implanting an intramedullary nail within bone, the intramedullary nail having a nail body elongate along a longitudinal nail axis, a spring member disposed within the nail body, and a shuttle joined with the spring member and defining an aperture that extends through the shuttle along an aperture axis angularly offset from the longitudinal nail axis, the aperture configured to receive a locking member for affixing the shuttle to bone, the instrument assembly comprising:
a nail inserter having an inserter body that defines a trailing end, an opposed leading end, and a cannulation extending from the trailing end to the leading end in a forward direction oriented along a central axis of the inserter body, wherein the leading end is configured to couple with a trailing end of the intramedullary nail;
an instrument comprising:
an elongate instrument body having a leading portion that defines engagement structures and is configured to extend through the cannulation of the nail inserter and into an opening at a trailing end of the shuttle such that the engagement structures of the elongate instrument body are positioned adjacent complementary engagement formations defined along a trailing portion of the shuttle;
a first force actuator configured to move the elongate instrument body relative to the nail inserter to cause the engagement structures to engage the complementary engagement formations of the shuttle, thereby attaching the instrument to the shuttle;
one or more transmission structures defined on an intermediate portion of the elongate instrument body; and
a second force actuator connectable to the intermediate portion of the elongate instrument body, wherein the second force actuator defines one or more complementary transmission formations configured to engage the one or more transmission structures, and the second force actuator is manipulatable to cause the one or more transmission formations to engage the one or more transmission structures in a manner to translate the elongate instrument body and the shuttle in unison in a rearward direction opposite the forward direction, thereby displacing the spring member so as to load a spring force in the spring member in the forward direction.

2. The instrument assembly of claim 1, wherein the engagement structures of the instrument comprise external threads on an exterior surface of the leading portion of the instrument body, wherein the external threads extend to a leading end of the instrument body, and the external threads are configured for threaded engagement with the complementary engagement formations of the shuttle.

3. The instrument assembly of claim 2, wherein the first force actuator comprises a knob configured to rotate the elongate instrument body about a central instrument axis of the elongate instrument body, thereby threadedly engaging the external threads of the exterior surface of the leading portion with the complementary engagement formations of the shuttle.

4. The instrument assembly of claim 1, wherein:
the one or more transmission structures comprise external threads;
the second force actuator comprises a knob having a knob body defining a leading knob end surface, a trailing knob end surface opposite the leading knob end surface, and a bore extending through the knob body from the trailing knob end surface to the leading knob end surface, wherein the one or more complementary transmission formations comprise internal threads defined along an inner surface of the knob body within the bore; and
the knob is configured such that:
the first knob end surface contacts the trailing end of the nail inserter while the internal threads of the knob engage the external threads on the intermediate portion of the elongate instrument body; and
rotation of the knob about a central instrument axis of the elongate instrument body translates the elongate instrument body and the shuttle in unison in the rearward direction relative to the nail inserter and the intermedullary nail.

5. The instrument assembly of claim 4, wherein the knob body defines a hole extending from an exterior surface of the knob body toward the central instrument axis, and the instrument assembly further comprises a wrench member configured for insertion into the hole of the knob, wherein the wrench member is configured for increasing manual torque applied to the knob for rotating the knob in the first rotational direction.

6. An intramedullary nailing system, comprising:
an intramedullary nail, comprising:
a nail body elongate along a longitudinal nail axis;
a spring member disposed within an interior channel defined by the nail body; and
a shuttle joined with the spring member within the interior channel, the shuttle defining at least one aperture extending through the shuttle along an aperture axis angularly offset from the longitudinal nail axis, the at least one aperture configured to receive a locking member for affixing the shuttle to bone;
a nail inserter having a leading end and a trailing end spaced from each other along a longitudinal direction, wherein the leading end is configured to couple with a trailing end of the intramedullary nail, the nail inserter having an inserter body that defines an inserter cannulation extending from the trailing end to the leading end in a forward direction oriented along a central axis of the inserter body, wherein the forward direction is oriented along the longitudinal direction; and
an instrument comprising:
an instrument body elongate along a central instrument axis and having a trailing portion, a leading portion spaced from the trailing portion in the forward direction, and an intermediate portion located longitudinally between the trailing and leading portions, the leading portion defining engagement structures and is configured to extend through the inserter cannulation and into engagement with the shuttle, the intermediate portion defining one or more transmission structures; and
a force actuator connectable to the intermediate portion of the elongate instrument body, the force actuator defining one or more complementary transmission formations configured to engage the one or more transmission structures, wherein the force actuator is manipulatable to cause the one or more transmission formations to engage the one or more transmission structures in a manner to translate the elongate instrument body and the shuttle in unison in a rearward direction opposite the forward direction, thereby displacing the spring member so as to load a spring force in the spring member in the forward direction.

7. The system of claim 6, wherein the spring member has a leading spring end and a trailing spring end spaced from each other along the longitudinal nail axis, the leading spring end is coupled to a leading portion of the shuttle, and the trailing spring end is configured to abut a stop member of the intramedullary nail as the shuttle is translated in the rearward direction so as to compress the spring member to load the spring force.

8. The intramedullary nailing system of claim 7, wherein the spring member is constructed of nitinol.

9. The intramedullary nailing system of claim 6, wherein the nail body defines at least one slot extending radially outward from the interior channel to an exterior surface of the nail body, wherein the at least one aperture of the shuttle is configured to align with the at least one slot when the shuttle has been translated in the rearward direction.

10. The intramedullary nailing system of claim 6, wherein:
the force actuator is a knob having a knob body that defines a trailing end surface, an opposed leading end surface, a central bore extending longitudinally from the trailing end surface to the leading end surface, and an interior surface defining internal threads within the central bore; and
the one or more transmission structures comprise external threads defined along the intermediate portion of the instrument body, wherein the internal threads of the knob are configured to threadedly engage the external threads of the intermediate portion such that rotation of the knob about the central instrument axis causes the translation.

11. The intramedullary nailing system of claim 10, wherein the leading end surface of the knob is configured to abut the trailing end of the nail inserter during the rotation in a manner preventing the knob from translating in the forward direction relative to the nail inserter and thereby forcing the translation in the rearward direction.

12. The intramedullary nailing system of claim 11, wherein the knob body defines an outer surface extending longitudinally from the trailing end surface to the leading end surface, the knob body further defining a hole extending from the outer knob surface toward the central instrument axis, and the instrument further comprises a wrench member insertable within the hole and configured to be manually manipulated to increase a torque applied to the knob for rotating the knob.

13. The intramedullary nailing system of claim 10, wherein the instrument further comprises another force actuator configured to move the elongate instrument body relative to the intramedullary nail to cause the engagement structures of the elongate instrument body to engage the complementary engagement formations of the shuttle, thereby attaching the instrument to the shuttle.

14. The intramedullary nailing system of claim 13, wherein:
the engagement structures of the instrument body comprise external threads extending to a leading end of the instrument body;
the complementary engagement formations of the shuttle are internal threads defined with a bore extending along a trailing portion of the shuttle; and
the another force actuator of the instrument is another knob, the another knob located on the trailing portion of the instrument body, wherein the another knob is rotatable to threadedly engage the external threads of the instrument body with the internal threads of the shuttle.

15. A method for preparing an intramedullary nail to provide bone compression, the method comprising:
attaching a leading portion of a nail inserter to a trailing portion of a nail body of an intramedullary nail, wherein the nail body is elongate along a longitudinal nail axis, the intramedullary nail comprising:
a spring member disposed within an interior channel defined by the nail body; and
a shuttle joined with the spring member within the interior channel, the shuttle defining at least one aperture configured to receive a locking member for affixing the shuttle to bone;
wherein the nail body defines a leading portion spaced from the trailing portion in a forward direction;
advancing an instrument body of an instrument through a longitudinal cannulation of the nail inserter an into engagement with the shuttle;
coupling a leading portion of the instrument body with the shuttle; and
manipulating a transmission member of the instrument relative to the instrument body in a manner translating the instrument body and the shuttle in unison in a rearward direction opposite the forward direction, thereby loading a spring force in the spring member in the forward direction, wherein the spring force is configured to drive the shuttle in the forward direction to provide the bone compression.

16. The method of claim 15, wherein the transmission member is a knob having internal threads in threaded engagement with external threads of the instrument body, and manipulating the transmission member comprises rotating the knob about a central instrument axis of the instrument body while a leading end surface of the knob contacts a trailing end surface of the nail inserter.

17. The method of claim 16, wherein the shuttle defines a bore having internal threads, the leading portion of the instrument body has external threads, and the step of coupling the leading portion of the instrument body with the shuttle comprises rotating a second knob of the instrument about the central instrument axis, thereby threadedly coupling the external threads of the leading portion of the instrument body with the internal threads of the shuttle.

18. A method of implanting the intramedullary nail of claim 15, the method comprising:
manipulating the nail inserter to thereby insert the intramedullary nail within a medullary canal of a bone in the forward direction;
inserting at least one distal locking member through the at least one aperture of the shuttle, thereby affixing the shuttle to adjacent distal bone material; and
inserting one or more proximal locking members through one or more associated locking holes defined by the leading portion of the nail body, thereby affixing the leading portion of the nail body to adjacent proximal bone material.

19. The method of claim 18, comprising uncoupling the instrument body from the shuttle, thereby causing the spring force to provide bone compression force between the distal bone material and the proximal bone material.
